(19) United States
(12) Patent Application Publication
(10) Pub. No.: US 2026/0256461 A1
(43) Pub. Date: Sep. 3, 2026

(54) CALIBRATION METHOD FOR AN ULTRASOUND PROBE ASSEMBLY, ULTRASOUND PROBE ASSEMBLY, AND ULTRASOUND IMAGING SYSTEM

(71) Applicant: GE Precision Healthcare LLC, Waukesha, WI (US)

(72) Inventors: Xin Ge, Wuxi (CN); Gengjie Du, Wuxi (CN); Weidong Yuan, Wuxi (CN); Ningmei Liu, Wuxi (CN)

(21) Appl. No.: 19/553,932

(22) Filed: Mar. 2, 2026

(30) Foreign Application Priority Data
Feb. 28, 2025 (CN) 202510231243.4

(51) Int. Cl. A61B 8/00; A61B 8/08; G01S 7/52

(52) U.S. Cl. CPC A61B 8/58; A61B 8/0841; A61B 8/4461; G01S 7/5205; A61B 2560/0223

(57) ABSTRACT

A calibration method for an ultrasound probe assembly including an ultrasound probe, a driving apparatus, a grid plate, and at least one puncture needle. The method includes acquiring, using the ultrasound probe, an ultrasound image including an image of the at least one puncture needle and a two-dimensional coordinate system, the two-dimensional coordinate system including at least one coordinate point corresponding to the at least one reference point; determining a deviation between the image of the at least one puncture needle and the at least one coordinate point; and controlling, in response to the deviation being greater than a first preset value, the driving apparatus to drive the ultrasound probe to move so as to cause the deviation to be less than or equal to the first preset value.

### CROSS-REFERENCE TO RELATED APPLICATION

This application claims priority to Chinese Patent Application No. 202510231243.4, which was file on Feb. 28, 2025 at the Chinese Patent Office. The entire contents of the above-listed application are incorporated by reference herein in their entirety.

### TECHNICAL FIELD

The present disclosure relates to the field of medical instrument calibration, and in particular, to a calibration method for an ultrasound probe assembly, an ultrasound probe assembly, and an ultrasound imaging system.

### BACKGROUND

An ultrasound imaging system is a medical device that utilizes the principle of ultrasonic wave propagation and reflection in human tissues to image. An ultrasound probe is the core component of the ultrasound imaging system. The principle thereof is to emit ultrasonic waves toward a tissue to be imaged of a patient, receive reflected ultrasonic waves, convert the received ultrasonic waves into electrical signals, and obtain ultrasound images on the basis of electrical signal processing. Doctors may determine a lesion location according to the ultrasound images, and move a puncture needle such that the image of the puncture needle in the ultrasound image is aligned with the lesion location, then use the puncture needle to obtain a tissue sample, and perform laboratory analysis on the obtained tissue sample to determine the patient's condition. In order to enable precise guidance of the puncture, a coordinate system is usually included in the ultrasound image, and the coordinates of the lesion within the coordinate system of the ultrasound image are determined to guide which reference point (or hole) on the grid plate the puncture needle should be inserted into. Obviously, the alignment relationship between the coordinates of the ultrasound image and the grid plate is crucial for the puncture. If the deviation between the two is too large, it can easily lead to medical failure.

Ultrasound probes may be manually calibrated by doctors based on experience. Manual calibration of ultrasound probes is inefficient and prone to large errors.

### SUMMARY OF THE INVENTION

The present disclosure provides a calibration method for an ultrasound probe assembly, an ultrasound probe assembly, and an ultrasound imaging system. The method can achieve automatic calibration of an ultrasound probe in the ultrasound probe assembly, improve the calibration efficiency of the ultrasound probe, and reduce the calibration error of the ultrasound probe.

According to one aspect of the present disclosure, a calibration method for an ultrasound probe assembly is provided. The ultrasound probe assembly comprises an ultrasound probe, a driving apparatus, a grid plate, and at least one puncture needle, the at least one puncture needle being inserted into at least one reference point on the grid plate. The method comprises: acquiring, using the ultrasound probe, an ultrasound image comprising an image of the at least one puncture needle and a two-dimensional coordinate system, the two-dimensional coordinate system comprising at least one coordinate point corresponding to the at least one reference point; determining a deviation between the image of the at least one puncture needle and the at least one coordinate point; and controlling, in response to the deviation being greater than a first preset value, the driving apparatus to drive the ultrasound probe to move so as to cause the deviation to be less than or equal to the first preset value.

In one possible implementation, the coordinates of the coordinate point corresponding to each reference point in the two-dimensional coordinate system are associated with the position of the reference point on the grid plate.

In one possible implementation, the coordinates of the coordinate point corresponding to each reference point in the two-dimensional coordinate system are inputted by a user.

In one possible implementation, the determining a deviation between the image of the at least one puncture needle and the at least one coordinate point comprises: identifying the image of the at least one puncture needle from the ultrasound image; and determining the deviation between the image of the at least one puncture needle and the at least one coordinate point according to a linear distance between the image of the at least one puncture needle and the coordinate point corresponding thereto in the two-dimensional coordinate system.

In one possible implementation, a manner in which the driving apparatus drives the ultrasound probe to move comprises at least one of translation and rotation.

In one possible implementation, the controlling, in response to the deviation being greater than a first preset value, the driving apparatus to drive the ultrasound probe to move comprises: determining operating parameters of the driving apparatus according to the coordinates of the image of each puncture needle and the coordinate point corresponding thereto in the two-dimensional coordinate system, wherein the operating parameters comprise at least one of a set of translation parameters and a set of rotation parameters, the translation parameters comprising a translation direction and a translation distance, and the rotation parameters comprising a rotation direction and a rotation angle; controlling the driving apparatus to drive, according to the operating parameters, the ultrasound probe to move; and determining again the deviation between the image of the at least one puncture needle and the at least one coordinate point.

In one possible implementation, the controlling, in response to the deviation being greater than a first preset value, the driving apparatus to drive the ultrasound probe to move comprises: controlling the driving apparatus to drive, according to preset operating parameters, the ultrasound probe to move, the operating parameters comprising at least one of a set of translation parameters and a set of rotation parameters, the translation parameters comprising a translation direction and a translation distance, and the rotation parameters comprising a rotation direction and a rotation angle; determining the deviation again; adjusting, when the deviation has increased, at least one of the preset operating parameters, and controlling the driving apparatus to drive, according to the adjusted operating parameter, the ultrasound probe to move; and maintaining, when the deviation has decreased but remains greater than the first preset value, the preset operating parameters unchanged, and continuing to control the driving apparatus to drive, according to the preset operating parameters, the ultrasound probe to move.

In one possible implementation, the at least one puncture needle comprises a first puncture needle and a second puncture needle, the at least one reference point comprises a first reference point and a second reference point, and the at least one coordinate point comprises a first coordinate point and a second coordinate point.

In one possible implementation, the at least one puncture needle comprises a first puncture needle and a second puncture needle, and the at least one coordinate point comprises a first coordinate point and a second coordinate point; and the determining operating parameters of the driving apparatus according to the coordinates of the image of each puncture needle and the coordinate point corresponding thereto in the two-dimensional coordinate system comprises: calculating a linear distance between the image of the first puncture needle and the image of the second puncture needle in the two-dimensional coordinate system according to the coordinates of the image of the first puncture needle and the image of the second puncture needle in the two-dimensional coordinate system, to obtain a first value; calculating a linear distance between the first coordinate point and the second coordinate point in the two-dimensional coordinate system according to the coordinates of the first coordinate point and the second coordinate point in the two-dimensional coordinate system, to obtain a second value; determining the translation distance according to a difference between the second value and the first value; and determining the translation direction according to a magnitude relationship between the second value and the first value.

In one possible implementation, the at least one puncture needle comprises a first puncture needle and a second puncture needle, and the at least one coordinate point comprises a first coordinate point and a second coordinate point; and the determining operating parameters of the driving apparatus according to the coordinates of the image of each puncture needle and the coordinate point corresponding thereto in the two-dimensional coordinate system comprises: determining a first connecting line between the image of the first puncture needle and the image of the second puncture needle in the two-dimensional coordinate system according to the coordinates of the image of the first puncture needle and the image of the second puncture needle in the two-dimensional coordinate system; determining a second connecting line between the first coordinate point and the second coordinate point in the two-dimensional coordinate system according to the coordinates of the first coordinate point and the second coordinate point in the two-dimensional coordinate system; determining the rotation angle according to an included angle between the first connecting line and the second connecting line; and determining the rotation direction according to a magnitude relationship between slopes of the first connecting line and the second connecting line.

According to another aspect of the present disclosure, an ultrasound probe assembly is provided, comprising an ultrasound probe, a driving apparatus, a grid plate, and at least one puncture needle, wherein the at least one puncture needle is inserted into at least one reference point on the grid plate; the ultrasound probe is configured to acquire an ultrasound image comprising an image of the at least one puncture needle and a two-dimensional coordinate system, the two-dimensional coordinate system comprising at least one coordinate point corresponding to the at least one reference point; and the driving apparatus is configured to drive the ultrasound probe to move so as to cause a deviation between the image of the at least one puncture needle and the at least one coordinate point to be less than or equal to a first preset value.

In one possible implementation, the driving apparatus comprises at least one of a translation motor and a rotation motor, the translation motor being configured to drive the ultrasound probe to translate, and the rotation motor being configured to drive the ultrasound probe to rotate.

In one possible implementation, the driving apparatus further comprises a guide rail, a first connecting member, and a second connecting member, the translation motor being connected to the guide rail by means of the first connecting member, the rotation motor being fixed to a sliding block on the guide rail, and the rotation motor being connected to the ultrasound probe by means of the second connecting member; the translation motor causes the rotation motor, the second connecting member, and the ultrasound probe to translate by means of translating the sliding block; and the rotation motor causes the ultrasound probe to rotate by means of rotating the second connecting member.

In one possible implementation, the driving apparatus further comprises a guide rail, a third connecting member, and a fourth connecting member, the rotation motor being connected to the guide rail by means of the third connecting member, the translation motor being connected to the guide rail by means of the fourth connecting member, and the ultrasound probe being fixed to a sliding block on the guide rail; the rotation motor causes the guide rail, the fourth connecting member, the sliding block, and the ultrasound probe to rotate by means of rotating the third connecting member; and the translation motor causes the ultrasound probe to translate by means of translating the sliding block.

According to a yet another aspect of the present disclosure, an ultrasound imaging system is provided, comprising: a processor; a memory, configured to store instructions executable by the processor; and an ultrasound probe assembly, comprising an ultrasound probe, a driving apparatus, a grid plate, and at least one puncture needle, the at least one puncture needle being inserted into at least one reference point on the grid plate; wherein the processor is configured to implement the described method when executing the instructions stored in the memory.

In one possible implementation, the processor is further configured to execute the instructions stored in the memory to implement: identifying, during an interventional procedure, a region of interest in an intraoperative ultrasound image acquired by the ultrasound probe, the intraoperative ultrasound image comprising an image of a tissue to be imaged and a two-dimensional coordinate system, and the two-dimensional coordinate system comprising a target coordinate point; and controlling the driving apparatus to drive, according to the coordinates of the region of interest and the target coordinate point in the two-dimensional coordinate system comprised by the intraoperative ultrasound image, the ultrasound probe to move.

In one possible implementation, the controlling the driving apparatus to drive, according to the coordinates of the region of interest and the target coordinate point in the two-dimensional coordinate system comprised by the intraoperative ultrasound image, the ultrasound probe to move comprises: determining operating parameters of the driving apparatus according to the coordinates of the region of interest and the target coordinate point in the two-dimensional coordinate system comprised by the intraoperative ultrasound image, wherein the operating parameters comprise at least one of a set of translation parameters and a set of rotation parameters, the translation parameters comprising a translation direction and a translation distance, and the rotation parameters comprising a rotation direction and a rotation angle; and causing the driving apparatus to operate according to the operating parameters.

In one possible implementation, the processor is further configured to execute the instructions stored in the memory to implement: causing, after controlling the driving apparatus to drive the ultrasound probe to move, the ultrasound probe to reacquire an intraoperative ultrasound image, and identifying a region of interest in the reacquired intraoperative ultrasound image; determining a distance between a center point of the region of interest and the target coordinate point in a two-dimensional coordinate system comprised by the reacquired intraoperative ultrasound image; and continuing to control, when the distance is greater than a second preset value, the driving apparatus to drive the ultrasound probe to move.

According to yet another aspect of the present disclosure, a calibration apparatus for an ultrasound probe assembly is provided. The ultrasound probe assembly comprises an ultrasound probe, a driving apparatus, a grid plate, and at least one puncture needle, the at least one puncture needle being inserted into at least one reference point on the grid plate. The apparatus comprises: an acquisition module, configured to acquire, using the ultrasound probe, an ultrasound image comprising an image of the at least one puncture needle and a two-dimensional coordinate system, the two-dimensional coordinate system comprising at least one coordinate point corresponding to the at least one reference point; a determination module, configured to determine a deviation between the image of the at least one puncture needle and the at least one coordinate point; and a control module, configured to control, in response to the deviation being greater than a first preset value, the driving apparatus to drive the ultrasound probe to move so as to cause the deviation to be less than or equal to the first preset value.

In one possible implementation, the coordinates of the coordinate point corresponding to each reference point in the two-dimensional coordinate system are associated with the position of the reference point on the grid plate.

In one possible implementation, the coordinates of the coordinate point corresponding to each reference point in the two-dimensional coordinate system are inputted by a user.

In one possible implementation, the determining a deviation between the image of the at least one puncture needle and the at least one coordinate point comprises: identifying the image of at least one puncture needle from the ultrasound image; and determining the deviation between the image of the at least one puncture needle and the at least one coordinate point according to a linear distance between the image of the at least one puncture needle and the coordinate point corresponding thereto in the two-dimensional coordinate system.

In one possible implementation, a manner in which the driving apparatus drives the ultrasound probe to move comprises at least one of translation and rotation.

In one possible implementation, the controlling, in response to the deviation being greater than a first preset value, the driving apparatus to drive the ultrasound probe to move comprises: determining operating parameters of the driving apparatus according to the coordinates of the image of each puncture needle and the coordinate point corresponding thereto in the two-dimensional coordinate system, wherein the operating parameters comprise at least one of a set of translation parameters and a set of rotation parameters, the translation parameters comprising a translation direction and a translation distance, and the rotation parameters comprising a rotation direction and a rotation angle; controlling the driving apparatus to drive, according to the operating parameters, the ultrasound probe to move; and determining again the deviation between the image of the at least one puncture needle and the at least one coordinate point.

In one possible implementation, the controlling, in response to the deviation being greater than a first preset value, the driving apparatus to drive the ultrasound probe to move comprises: controlling the driving apparatus to drive, according to preset operating parameters, the ultrasound probe to move, wherein the operating parameters comprise at least one of a set of translation parameters and a set of rotation parameters, the translation parameters comprising a translation direction and a translation distance, and the rotation parameters comprising a rotation direction and a rotation angle; determining the deviation again; adjusting, when the deviation has increased, at least one of the preset operating parameters, and controlling the driving apparatus to drive, according to the adjusted operating parameter, the ultrasound probe to move; and maintaining, when the deviation has decreased but remains greater than the first preset value, the preset operating parameters unchanged, and continuing to control the driving apparatus to drive, according to the preset operating parameters, the ultrasound probe to move.

In one possible implementation, the at least one puncture needle comprises a first puncture needle and a second puncture needle, the at least one reference point comprises a first reference point and a second reference point, and the at least one coordinate point comprises a first coordinate point and a second coordinate point.

In one possible implementation, the at least one puncture needle comprises a first puncture needle and a second puncture needle, and the at least one coordinate point comprises a first coordinate point and a second coordinate point; and the determining operating parameters of the driving apparatus according to the coordinates of the image of each puncture needle and the coordinate point corresponding thereto in the two-dimensional coordinate system comprises: calculating a linear distance between the image of the first puncture needle and the image of the second puncture needle in the two-dimensional coordinate system according to the coordinates of the image of the first puncture needle and the image of the second puncture needle in the two-dimensional coordinate system, to obtain a first value; calculating a linear distance between the first coordinate point and the second coordinate point in the two-dimensional coordinate system according to the coordinates of the first coordinate point and the second coordinate point in the two-dimensional coordinate system, to obtain a second value; determining a translation distance according to a difference between the second value and the first value; and determining a translation direction according to a magnitude relationship between the second value and the first value.

In one possible implementation, the at least one puncture needle comprises a first puncture needle and a second puncture needle, and the at least one coordinate point comprises a first coordinate point and a second coordinate point; and the determining operating parameters of the driving apparatus according to the coordinates of the image of each puncture needle and the coordinate point corresponding thereto in the two-dimensional coordinate system comprises: determining a first connecting line between the image of the first puncture needle and the image of the second puncture needle in the two-dimensional coordinate system according to the coordinates of the image of the first puncture needle and the image of the second puncture needle in the two-dimensional coordinate system; determining a second connecting line between the first coordinate point and the second coordinate point in the two-dimensional coordinate system according to the coordinates of the first coordinate point and the second coordinate point in the two-dimensional coordinate system; determining a rotation angle according to an included angle between the first connecting line and the second connecting line; and determining a rotation direction according to a magnitude relationship between slopes of the first connecting line and the second connecting line.

According to another aspect of the present disclosure, provided is a non-volatile computer-readable storage medium, having computer program instructions stored thereon, wherein the computer program instructions, when executed by a processor, implement the above method.

According to another aspect of the present disclosure, provided is a computer program product, comprising computer-readable code or a non-volatile computer-readable storage medium bearing computer-readable code, wherein when the computer-readable code is run in a processor of an electronic device, the processor in the electronic device performs the above method.

According to the calibration method for an ultrasound probe assembly in the embodiments of the present disclosure, wherein the ultrasound probe assembly comprises an ultrasound probe, a driving apparatus, a grid plate, and at least one puncture needle, the at least one puncture needle being inserted into at least one reference point on the grid plate, by means of acquiring, using the ultrasound probe, an ultrasound image comprising an image of the at least one puncture needle and a two-dimensional coordinate system, the two-dimensional coordinate system comprising at least one coordinate point corresponding to the at least one reference point, both the image of the puncture needle and the coordinate point corresponding to the puncture needle are simultaneously present in the ultrasound image; by means of determining a deviation between the image of the at least one puncture needle and the at least one coordinate point, it can be determined that in an actual scenario, after the coordinates of a lesion in the ultrasound image are determined, the puncture needle can accurately arrive at the lesion when positioned at a location corresponding to the coordinates on the grid plate, e.g., when the deviation is too large, it can be determined that the correspondence between the grid plate and the coordinate position of the ultrasound image is insufficient; and by means of controlling, in response to the deviation being greater than a first preset value, the driving apparatus to drive the ultrasound probe to move so as to cause the deviation to be less than or equal to the first preset value, in the actual scenario, the coordinate points in the ultrasound image and the reference points on the grid plate may be considered as having a good correspondence, thereby achieving calibration of the ultrasound probe. The method can achieve automatic calibration of the ultrasound probe in the ultrasound probe assembly, eliminating the need to manually calibrate the ultrasound probe, and improving the calibration efficiency of the ultrasound probe; and the comparison between the deviation and the first preset value is more accurate, thereby reducing the calibration error of the ultrasound probe.

Other features and aspects of the present disclosure will become apparent from the following detailed description of exemplary embodiments provided with reference to the accompanying drawings.

### BRIEF DESCRIPTION OF THE DRAWINGS

The accompanying drawings, incorporated in and constituting a part of the specification, illustrate exemplary embodiments, features, and aspects of the present disclosure together with the specification, and serve to explain the principles of the present disclosure.

FIG. 1 shows an exemplary application scenario of a calibration method for an ultrasound probe assembly according to an embodiment of the present disclosure.

FIG. 2 shows an exemplary application scenario of an ultrasound probe assembly according to an embodiment of the present disclosure.

FIG. 3 shows a schematic flowchart of a calibration method for an ultrasound probe assembly according to an embodiment of the present disclosure.

FIG. 4 shows a schematic diagram of inserting a puncture needle into a reference point on a grid plate according to an embodiment of the present disclosure.

FIG. 5 shows a schematic diagram of an ultrasound image acquired when an ultrasound probe is calibrated according to an embodiment of the present disclosure.

FIG. 6 shows a schematic diagram of the structure of a driving apparatus according to an embodiment of the present disclosure.

FIG. 7 shows a schematic diagram of the structure of a driving apparatus according to an embodiment of the present disclosure.

FIG. 8 shows a schematic diagram of the structure of a calibration apparatus for an ultrasound probe assembly according to an embodiment of the present disclosure.

FIG. 9 shows a schematic diagram of the structure of an ultrasound imaging system according to an embodiment of the present disclosure.

### DETAILED DESCRIPTION

Various exemplary embodiments, features, and aspects of the present disclosure will be described in detail below with reference to the accompanying drawings. The same reference numerals in the drawings represent elements having the same or similar functions. While various aspects of the embodiments are illustrated in the accompanying drawings, the accompanying drawings are not necessarily drawn to scale unless specifically stated.

The word “exemplary” dedicated herein means “used as an example or an embodiment, or illustrative”. Any of the embodiments illustrated herein as “exemplary” is not necessarily interpreted as being superior to or better than other embodiments.

In addition, in order to better illustrate the present disclosure, numerous specific details are given in the detailed description below. It should be understood by those skilled in the art that the present disclosure can be implemented without some of the specific details. In some examples, the methods, means, elements, and circuits well known to those skilled in the art are not described in detail in order to highlight the main idea of the present disclosure.

The present disclosure provides a calibration method for an ultrasound probe assembly, an ultrasound probe assembly, and an ultrasound imaging system. The method can achieve automatic calibration of an ultrasound probe in the ultrasound probe assembly, improve the calibration efficiency of the ultrasound probe, and reduce the calibration error of the ultrasound probe.

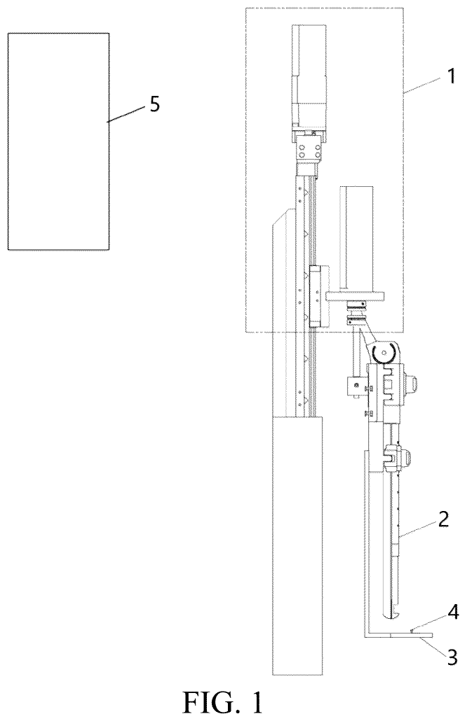

FIG. 1 shows an exemplary application scenario of a calibration method for an ultrasound probe assembly according to an embodiment of the present disclosure.

As shown in FIG. 1, the ultrasound probe assembly may include an ultrasound probe 2, a driving apparatus 1, a grid plate 3, and at least one puncture needle 4. The grid plate 3 may include a plurality of reference points. The ultrasound probe 2 may be configured to acquire an ultrasound image, and the driving apparatus 1 may be configured to drive the ultrasound probe 2 to move. The driving apparatus 1 may communicate with a processor 5. The calibration method for an ultrasound probe assembly according to the embodiments of the present disclosure may be executed by the processor. The processor may, by issuing a control signal, control the driving apparatus to drive the ultrasound probe to move. The embodiments of the present disclosure impose no limitation on the specific control manner of the driving apparatus.

Before a patient's tissue to be imaged is prepared to be sampled, the ultrasound probe needs to be calibrated. Before the calibration of the ultrasound probe starts, the at least one puncture needle may be inserted into at least one reference point on the grid plate, and a reflector, such as water, may be placed in an ultrasonic wave emitting direction of the ultrasound probe. Subsequently, the processor may execute the calibration method for the ultrasound probe assembly, control the ultrasound probe to emit ultrasonic waves, and obtain an ultrasound image according to reflected echoes. The ultrasound image may include an image of the puncture needle, and a coordinate point corresponding to the reference point in which the puncture needle is inserted. The processor may determine a control method for the driving apparatus according to the ultrasound image, generate a corresponding control signal, and transmit the control signal to the driving apparatus, causing the driving apparatus to drive the ultrasound probe to move, thereby completing calibration of the ultrasound probe.

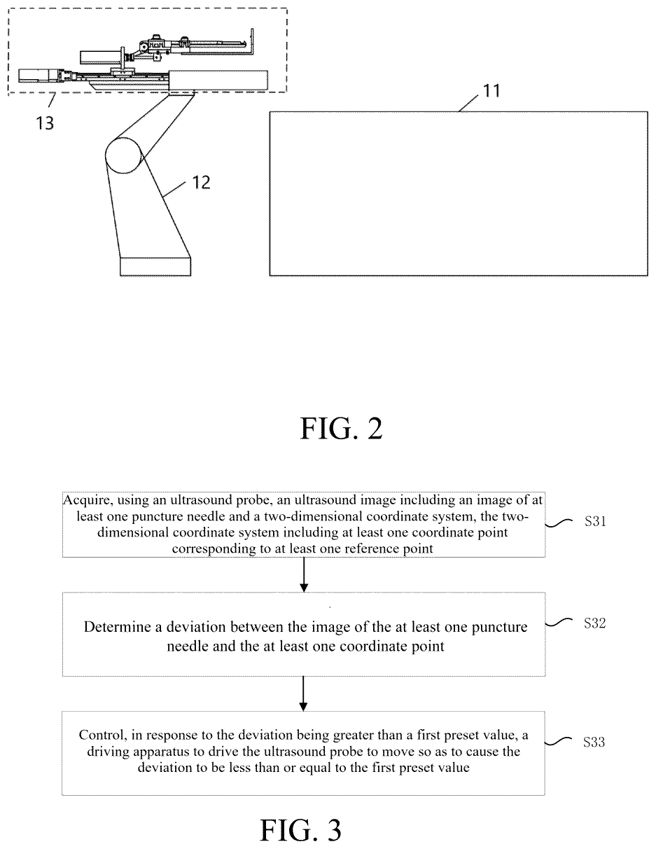

FIG. 2 shows an exemplary application scenario of an ultrasound probe assembly according to an embodiment of the present disclosure.

As shown in FIG. 2, after calibration of the ultrasound probe is completed, an entire ultrasound probe assembly 13 may be moved to an operating table 11 using a robotic arm 12. After an interventional procedure begins, an ultrasound image of a tissue to be imaged of a patient may be acquired using the ultrasound probe, a lesion location may be determined in the ultrasound image, the position of the ultrasound probe assembly may be adjusted such that the puncture needle is aligned with the lesion location in the ultrasound image, and then a tissue sample at a corresponding position may be acquired using the puncture needle. The robotic arm may be controlled by a processor (not shown), and the embodiments of the present disclosure impose no limitation on a specific control manner for the robotic arm.

During the interventional procedure, when an image of the tissue to be imaged is not located in a central region of the ultrasound image, the processor may also output a control signal to the driving apparatus to control the driving apparatus to move the ultrasound probe, so as to cause the image of the tissue to be imaged to be located in the central region of the ultrasound image, thereby enabling a doctor to determine the lesion location more accurately by means of the ultrasound image.

FIG. 3 shows a schematic flowchart of a calibration method for an ultrasound probe assembly according to an embodiment of the present disclosure.

In one possible implementation, the ultrasound probe assembly includes an ultrasound probe, a driving apparatus, a grid plate, and at least one puncture needle. The at least one puncture needle is inserted into at least one reference point on the grid plate. For the structure of the ultrasound probe assembly, refer to FIG. 1.

As shown in FIG. 3, the method includes:

Step S31: acquiring, using the ultrasound probe, an ultrasound image including an image of the at least one puncture needle and a two-dimensional coordinate system, the two-dimensional coordinate system including at least one coordinate point corresponding to the at least one reference point;

Step S32: determining a deviation between the image of the at least one puncture needle and the at least one coordinate point; and

Step S33: controlling, in response to the deviation being greater than a first preset value, the driving apparatus to drive the ultrasound probe to move so as to cause the deviation to be less than or equal to the first preset value.

For example, in step S31, for an exemplary manner of acquiring the ultrasound image using the ultrasound probe, refer to the related description for FIG. 1. The grid plate may include a plurality of reference points distributed in a grid, and the at least one puncture needle may be inserted into at least one reference point on the grid plate. Correspondingly, the ultrasound image may also include a two-dimensional coordinate system, the two-dimensional coordinate system may include at least one coordinate point, and the coordinate points correspond one-to-one to the reference points where the puncture needles are inserted.

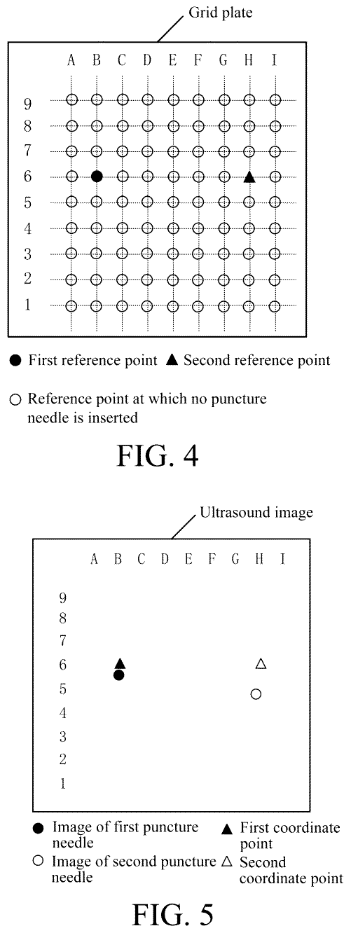

FIG. 4 shows a schematic diagram of inserting a puncture needle into a reference point on a grid plate according to an embodiment of the present disclosure. FIG. 5 shows a schematic diagram of an ultrasound image acquired when an ultrasound probe is calibrated according to an embodiment of the present disclosure.

As shown in FIG. 4, coordinates of the reference point on the grid plate may be (x, y), where x ∈ {1, 2, 3, 4, 5, 6, 7, 8, 9} and y ∈ {A, B, C, D, E, F, G, H, I}. Assuming that the at least one puncture needle includes a first puncture needle and a second puncture needle, the first puncture needle is inserted into a first reference point (6, B) on the grid plate, and the second puncture needle is inserted into a second reference point (6, H) on the grid plate. As shown in FIG. 5, a two-dimensional coordinate system in the ultrasound image may include a first coordinate point and a second coordinate point, wherein the coordinates of the first coordinate point are (6, B), which corresponds to the first reference point, and the coordinates of the second coordinate point are (6, H), which corresponds to the second reference point.

The ultrasound image may further include an image of the at least one puncture needle. The ultrasound probe includes an ultrasonic transducer. The puncture needle can be imaged only when the puncture needle passes through an imaging plane of the ultrasonic transducer. Thus, the image of the puncture needle in the ultrasound image appears as a point. As shown in FIG. 5, when the ultrasound image includes the first coordinate point and the second coordinate point, the ultrasound image may include an image of the first puncture needle and an image of the second puncture needle. In this case, each puncture needle corresponds to the insertion reference point thereof, and also to the coordinate point corresponding to the reference point. Therefore, the image of each puncture needle corresponds to the coordinate point corresponding to that puncture needle.

It may be considered that in an actual scenario, a lesion location should lie on an extension line of the puncture needle inserted into the reference point, and at the same time, the lesion location in the ultrasound image is located at the coordinate point corresponding to the reference point. That is, there should be a correspondence between the coordinate points and the reference points. When a puncture needle is inserted into a certain reference point, the image of the puncture needle appears near the coordinate point corresponding to the reference point. Therefore, conversely, when a doctor observes a lesion in the ultrasound image, the reference point on the grid plate at which the puncture needle needs to be inserted can be determined according to the coordinate point at which the lesion is located in the ultrasound image. It may be understood that ensuring the correspondence of positional relationships between the reference points and the corresponding coordinate points is crucial.

It should be understood that if the puncture needle has been inserted into the reference point at the time of calibration, the image of the puncture needle should accordingly be highly coincident with the position of the coordinate point corresponding to the reference point on the ultrasound image. If the image of the puncture needle is not highly coincident with the position of the coordinate point corresponding to the reference point on the ultrasound image, calibration is required. Therefore, after the ultrasound image including the image of the puncture needle and the coordinate point has been acquired, in step S32, a deviation between the image of the at least one puncture needle and the at least one coordinate point may be determined based on the ultrasound image. The deviation may directly reflect the deviation that would exist in an actual operation between the puncture needle and the lesion location after the reference point at which the puncture needle needs to be inserted is determined based on the lesion location in the ultrasound image and the corresponding coordinates thereof in an actual scenario. The embodiments of the present disclosure impose no limitation on the specific implementation of step S32, and an exemplary implementation of step S32 is provided later.

A first preset value may be predetermined. When the deviation between the image of the at least one puncture needle and the at least one coordinate point is greater than the first preset value, the deviation is considered unacceptable, and when the deviation between the image of the at least one puncture needle and the at least one coordinate point is less than or equal to the first preset value, the deviation is considered acceptable. The embodiments of the present disclosure impose no limitation on the specific numerical value of the first preset value, as long as the numerical value meets application scenario requirements.

The deviation calculated in step S32 may be compared with the first preset value. If the deviation is greater than the first preset value, step S33 is executed to control the driving apparatus to drive the ultrasound probe to move so as to cause the deviation to be less than or equal to the first preset value. The manner of driving the ultrasound probe to move may be determined first, and then the ultrasound probe driven to move, or the manner of driving the ultrasound probe to move may be determined and adjusted in the process of driving the ultrasound probe to move. The embodiments of the present disclosure impose no limitation on the specific implementation of step S33, as long as the deviation between the image of the at least one puncture needle and the at least one coordinate point is ultimately reduced below the first preset value. An exemplary implementation of step S33 is provided later.

According to the calibration method for an ultrasound probe assembly in the embodiments of the present disclosure, wherein the ultrasound probe assembly includes an ultrasound probe, a driving apparatus, a grid plate, and at least one puncture needle, the at least one puncture needle being inserted into at least one reference point on the grid plate, by means of acquiring, using the ultrasound probe, an ultrasound image including an image of the at least one puncture needle and a two-dimensional coordinate system, the two-dimensional coordinate system including at least one coordinate point corresponding to the at least one reference point, both the image of the puncture needle and the coordinate point corresponding to the puncture needle are simultaneously present in the ultrasound image; by means of determining a deviation between the image of the at least one puncture needle and the at least one coordinate point, the deviation between the puncture needle after puncturing and a lesion can be determined after the reference point at which the puncture needle needs to be inserted is determined using the lesion location in the ultrasound image and the corresponding coordinates thereof in an actual scenario; and by means of controlling, in response to the deviation being greater than the first preset value, the driving apparatus to drive the ultrasound probe to move so as to cause the deviation to be less than or equal to the first preset value, the coordinate system of the grid plate and the coordinate system of the ultrasound image may be considered as having a good correspondence, and the lesion coordinates in the ultrasound image can accurately provide a prompt for the reference point at which the puncture needle is inserted, thereby achieving the calibration of the ultrasound probe. The method can achieve automatic calibration of the ultrasound probe in the ultrasound probe assembly, eliminating the need to manually calibrate the ultrasound probe, and improving the calibration efficiency of the ultrasound probe; and the comparison between the deviation and the first preset value is more accurate, thereby reducing the calibration error of the ultrasound probe.

In one possible implementation, the coordinates of the coordinate point corresponding to each reference point in the two-dimensional coordinate system are associated with the position of the reference point on the grid plate.

For example, it is possible to enable each reference point on the grid plate to have a unique corresponding coordinate point in the two-dimensional coordinate system, and pre-store the correspondence between the reference points on the grid plate and the coordinate points in the two-dimensional coordinate system. Since the positions of the reference points on the grid plate are different, when step S31 is executed to acquire the ultrasound image, the coordinate point corresponding to the reference point may be found among the preset correspondences according to the position of the reference point at which the puncture needle is inserted on the grid plate. Taking FIG. 4 and FIG. 5 as examples, the coordinates of the first coordinate point corresponding to the first reference point in the two-dimensional coordinate system may be the same as the coordinates of the first reference point on the grid plate.

In this way, the coordinate point corresponding to the reference point at which the puncture needle is inserted can also be automatically determined, thereby further increasing calibration efficiency.

In one possible implementation, the coordinates of the coordinate point corresponding to each reference point in the two-dimensional coordinate system are inputted by a user.

For example, a doctor may determine, according to the position of the reference point at which the puncture needle is inserted on the grid plate, the coordinate point corresponding to the reference point, and manually input the coordinates of the coordinate point. A processor may add a corresponding coordinate point in the ultrasound image according to the externally inputted coordinates.

In this way, data storage costs can be reduced, and the means of positioning the coordinate points corresponding to the reference points is more flexible.

An exemplary implementation of step S32 is described below.

In one possible implementation, step S32 includes:

Identifying the image of the at least one puncture needle from the ultrasound image; and

Determining the deviation between the image of the at least one puncture needle and the at least one coordinate point according to a linear distance between the image of the at least one puncture needle and the coordinate point corresponding thereto in the two-dimensional coordinate system.

For example, when step S32 is executed, the image of the at least one puncture needle may be first identified from the ultrasound image. The image of the puncture needle appears as a dot in the ultrasound image, and typically has high brightness. Therefore, in one example, points having brightness greater than a preset threshold may be identified from a region near the coordinate point in the ultrasound image, and a point having the highest brightness is selected from the identified points as the image of the puncture needle corresponding to the coordinate point. The embodiments of the present disclosure impose no limitation on the specific implementation of identifying the image of the at least one puncture needle from the ultrasound image.

The respective linear distances between images of puncture needles and coordinate points corresponding thereto in the two-dimensional coordinate system may be statistically calculated. In one example, a maximum value of the respective linear distances between the images of puncture needles and the coordinate points corresponding thereto in the two-dimensional coordinate system may be calculated as the deviation between the image of the at least one puncture needle and the at least one coordinate point.

Those skilled in the art should understand that an average value/variance/standard deviation/weighted sum result, etc., of the linear distances between the images of respective puncture needles and coordinate points corresponding thereto in the two-dimensional coordinate system may also be calculated as the deviation between the image of the at least one puncture needle and the at least one coordinate point. The embodiments of the present disclosure impose no limitation on the specific implementation of calculating the deviation.

In this way, the deviation between the image of the at least one puncture needle and the at least one coordinate point can be calculated.

In one possible implementation, a manner in which the driving apparatus drives the ultrasound probe to move includes at least one of translation and rotation.

For example, the driving apparatus may include at least one of a translation motor and a rotation motor. Accordingly, when a translation motor is included, the manner in which the driving apparatus drives the ultrasound probe to move includes translation, and when a rotation motor is included, the manner of driving the ultrasound probe to move includes rotation.

In this way, the manner in which the driving apparatus drives the ultrasound probe becomes more flexible.

An exemplary implementation of step S33 is described below.

In one possible implementation, step S33 includes:

Determining operating parameters of the driving apparatus according to the coordinates of the image of each puncture needle and the coordinate point corresponding thereto in the two-dimensional coordinate system, wherein the operating parameters include at least one of a set of translation parameters and a set of rotation parameters, the translation parameters including a translation direction and a translation distance, and the rotation parameters including a rotation direction and a rotation angle;

Controlling the driving apparatus to drive, according to the operating parameters, the ultrasound probe to move; and

Determining again the deviation between the image of the at least one puncture needle and the at least one coordinate point.

For example, the processor may first determine the operating parameters of the driving apparatus, and then control the driving apparatus to drive, according to the operating parameters, the ultrasound probe to move.

As described above, the manner in which the driving apparatus drives the ultrasound probe to move includes at least one of translation and rotation, and correspondingly, the operating parameters of the driving apparatus that can be determined may also include at least one of a set of translation parameters and a set of rotation parameters. The translation parameters include a translation direction and a translation distance, and the rotation parameters include a rotation direction and a rotation angle.

If the processor determines that the deviation between the image of the at least one puncture needle and the at least one coordinate point may be less than or equal to the first preset value after the ultrasound probe is driven to translate by a distance B1 in a direction A1, then A1 may be determined as the translation direction, and B1 may be determined as the translation distance, without determining rotation parameters. Similarly, if the processor determines that the deviation between the image of the at least one puncture needle and the at least one coordinate point may be less than or equal to the first preset value after the ultrasound probe is driven to rotate by an angle D1 in a direction C1, then C1 may be determined as the rotation direction, and D1 may be determined as the rotation angle, without determining translation parameters. If the processor determines that the deviation between the image of the at least one puncture needle and the at least one coordinate point may be less than or equal to the first preset value after the ultrasound probe is driven to translate by a distance B2 in a direction A2 and rotate by an angle D2 in a direction C2, then A2 may be determined as the translation direction, B2 may be determined as the translation distance, C2 may be determined as the rotation direction, and D2 may be determined as the rotation angle.

In one example, translation directions may include a direction approaching the grid plate and a direction away from the grid plate. Rotation directions may include a clockwise direction and a counterclockwise direction. Those skilled in the art should understand that the ultrasound probe is located in three-dimensional space, so when the manner of connecting the driving apparatus and the ultrasound probe meets the requirements of the application scenario, the driving apparatus may drive the ultrasound probe to translate and rotate in any direction in three-dimensional space. The embodiments of the present disclosure impose no limitation on the specific translation direction and rotation direction.

In one example, the operating parameters of the driving apparatus may be determined by the coordinates of the image of each puncture needle and the coordinate point corresponding thereto in the two-dimensional coordinate system. An example for determining the operating parameters of the driving apparatus when two puncture needles are inserted into the grid plate is provided below.

In one possible implementation, the at least one puncture needle includes a first puncture needle and a second puncture needle, the at least one reference point includes a first reference point and a second reference point, and the at least one coordinate point includes a first coordinate point and a second coordinate point.

For example, calibration may be achieved using two puncture needles. Referring to FIG. 4 and FIG. 5, the at least one reference point includes a first reference point and a second reference point, and the at least one coordinate point includes a first coordinate point and a second coordinate point. The at least one puncture needle may include a first puncture needle and a second puncture needle, the first puncture needle being inserted into the first reference point and the second puncture needle being inserted into the second reference point.

In one possible implementation, determining operating parameters of the driving apparatus according to the coordinates of the image of each puncture needle and the coordinate point corresponding thereto in the two-dimensional coordinate system includes:

Calculating a linear distance between the image of the first puncture needle and the image of the second puncture needle in the two-dimensional coordinate system according to the coordinates of the image of the first puncture needle and the image of the second puncture needle in the two-dimensional coordinate system, to obtain a first value;

Calculating a linear distance between the first coordinate point and the second coordinate point in the two-dimensional coordinate system according to the coordinates of the first coordinate point and the second coordinate point in the two-dimensional coordinate system, to obtain a second value;

Determining a translation distance according to a difference between the second value and the first value; and

Determining a translation direction according to a magnitude relationship between the second value and the first value.

For example, under ideal conditions, the image of the first puncture needle coincides with the first coordinate point, the image of the second puncture needle coincides with the second coordinate point, and the linear distance between the images of the first puncture needle and the second puncture needle is the same as the linear distance between the first coordinate point and the second coordinate point. When there is a deviation between the image of the at least one puncture needle and the at least one coordinate point, the linear distance between the images of the first puncture needle and the second puncture needle may be the same as or different from the linear distance between the first coordinate point and the second coordinate point.

Assuming that the linear distance between the image of the first puncture needle and the image of the second puncture needle can be changed by translating the ultrasound probe, when the positions of the first puncture needle and the second puncture needle are unchanged, the correlation between the translation distance of the ultrasound probe and the amount of change in the linear distance between the image of the first puncture needle and the image of the second puncture needle can be predetermined. The linear distance between the image of the first puncture needle and the image of the second puncture needle may be calculated according to the coordinates of the image of the first puncture needle and the image of the second puncture needle in the two-dimensional coordinate system, to obtain the first value, and the linear distance between the first coordinate point and the second coordinate point may be calculated according to the coordinates of the first coordinate point and the second coordinate point in the two-dimensional coordinate system, to obtain the second value. The translation distance is determined according to a difference between the second value and the first value, and the predetermined correlation between the translation distance of the ultrasound probe and the amount of change in the linear distance between the image of the first puncture needle and the image of the second puncture needle.

The correlation between the translation distance of the ultrasound probe and the amount of change in the linear distance between the image of the first puncture needle and the image of the second puncture needle may be determined based on the prior art, and details are not described again herein.

In one example, the translation of the ultrasound probe in the direction approaching to the grid plate may cause the first value to increase. Since the second value is unchanged, when the second value is greater than the first value, the first value is considered too small, and the direction approaching to the grid plate may be determined as the translation direction. When the second value is less than the first value, the first value is considered too large, and the direction away from the grid plate may be determined as the translation direction. When the second value is equal to the first value, the translation distance and the translation direction do not need to be determined.

In one possible implementation, determining operating parameters of the driving apparatus according to the coordinates of the image of each puncture needle and the coordinate point corresponding thereto in the two-dimensional coordinate system includes:

Determining a first connecting line between the image of the first puncture needle and the image of the second puncture needle in the two-dimensional coordinate system according to the coordinates of the image of the first puncture needle and the image of the second puncture needle in the two-dimensional coordinate system;

Determining a second connecting line between the first coordinate point and the second coordinate point in the two-dimensional coordinate system according to the coordinates of the first coordinate point and the second coordinate point in the two-dimensional coordinate system;

Determining a rotation angle according to an included angle between the first connecting line and the second connecting line; and

Determining a rotation direction according to a magnitude relationship between slopes of the first connecting line and the second connecting line.

For example, under ideal conditions, the image of the first puncture needle coincides with the first coordinate point, the image of the second puncture needle coincides with the second coordinate point, a slope of the connecting line between the image of the first puncture needle and the image of the second puncture needle is the same as a slope of the connecting line between the first coordinate point and the second coordinate point, and an included angle between the two connecting lines is 0. When there is a deviation between the image of the at least one puncture needle and the at least one coordinate point, the slope of the connecting line between the image of the first puncture needle and the image of the second puncture needle may be the same as or different from the slope of the connecting line between the first coordinate point and the second coordinate point, and the included angle between the connecting lines may be greater than or equal to 0.

Assuming that the slope of the connecting line between the image of the first puncture needle and the image of the second puncture needle can be changed by means of rotating the ultrasound probe, when the positions of the first puncture needle and the second puncture needle are unchanged, the correlation between the rotation angle of the ultrasound probe and the slope of the connecting line between the image of the first puncture needle and the image of the second puncture needle can be predetermined. The first connecting line between the image of the first puncture needle and the image of the second puncture needle may be determined according to the coordinates of the image of the first puncture needle and the image of the second puncture needle in the two-dimensional coordinate system, and the second connecting line between the first coordinate point and the second coordinate point may be determined according to the coordinates of the first coordinate point and the second coordinate point in the two-dimensional coordinate system. The rotation angle is determined according to the included angle between the first connecting line and the second connecting line, and the predetermined correlation between the rotation angle of the ultrasound probe and an amount of change in the slope of the image of the first puncture needle and the image of the second puncture needle.

The correlation between the rotation angle of the ultrasound probe and the amount of change in the slope of the image of the first puncture needle and the image of the second puncture needle may be determined based on the prior art, and details are not described again herein.

In one example, rotation of the ultrasound probe in the clockwise direction may cause the slope of the first connecting line to decrease. Since the slope of the second connecting line is unchanged, when the slope of the second connecting line is greater than the slope of the first connecting line, the slope of the first connecting line may be considered too small, and the clockwise direction may be determined as the rotation direction. When the slope of the second connecting line is less than the slope of the first connecting line, the slope of the second connecting line may be considered too large, and the counterclockwise direction may be determined as the rotation direction. When the slope of the second connecting line is equal to the slope of the first connecting line, the rotation angle and the rotation direction do not need to be determined.

It should be understood that, in actual application scenarios, more or fewer puncture needles may be used to implement calibration. The embodiments of the present disclosure impose no limitation on the specific number of puncture needles used during calibration.

Those skilled in the art should understand that the above manner of determining the operating parameters of the driving apparatus is merely exemplary, and in an actual scenario, the operating parameters of the driving apparatus may be determined in other manners based on the prior art. The embodiments of the present disclosure impose no limitation on the specific manner of determining the operating parameters of the driving apparatus.

Under ideal conditions, after the driving apparatus drives, according to the determined operating parameters, the ultrasound probe to move, the deviation between the image of the at least one puncture needle and the at least one coordinate point should be less than or equal to the first preset value. Considering that there may be an error when the driving apparatus drives the ultrasound probe to move in an actual scenario, after the driving apparatus drives the ultrasound probe to move, step S32 may be executed again to determine the deviation between the image of the at least one puncture needle and the at least one coordinate point, then the deviation is compared with the first preset value, and if the deviation is still greater than the first preset value, step S33 is executed again until the deviation is less than the first preset value. In this way, the accuracy of the calibration of the ultrasound probe can be ensured.

An exemplary implementation of step S33 is described below.

In one possible implementation, step S33 includes:

Controlling the driving apparatus to drive, according to preset operating parameters, the ultrasound probe to move, wherein the operating parameters include at least one of a set of translation parameters and a set of rotation parameters, the translation parameters including a translation direction and a translation distance, and the rotation parameters including a rotation direction and a rotation angle;

Determining the deviation again;

Adjusting, when the deviation has increased, at least one of the preset operating parameters, and controlling the driving apparatus to drive, according to the adjusted operating parameter, the ultrasound probe to move; and

Maintaining, when the deviation has decreased but remains greater than the first preset value, the preset operating parameters unchanged, and continuing to control the driving apparatus to drive, according to the preset operating parameters, the ultrasound probe to move.

For example, the operating parameters of the driving apparatus may be preset. As described above, the manner in which the driving apparatus drives the ultrasound probe to move includes at least one of translation and rotation, and correspondingly, the preset operating parameters of the driving apparatus may also include at least one of a set of translation parameters and a set of rotation parameters. The translation parameters include a translation direction and a translation distance, and the rotation parameters include a rotation direction and a rotation angle. Examples of the translation direction and the rotation direction have been described above, and details will not be described again here.

The driving apparatus may drive, according to the preset operating parameters, the ultrasound probe to move, and after the ultrasound probe is driven to move, step S32 may be executed again to determine the deviation. Compared to the deviation determined in the previous execution of step S32, the currently determined deviation may increase or decrease.

When the currently determined deviation increases compared to the deviation determined in the previous execution of step S32, it indicates that the deviation cannot be reduced by continuing to use the preset operating parameters to move the ultrasound probe, and at least one of the preset operating parameters may be adjusted. For example, when the preset operating parameters include the translation direction, the translation direction is adjusted to be opposite to a preset translation direction, and when the preset operating parameters include the rotation direction, the rotation direction is adjusted to be opposite to a preset rotation direction. Adjusting the operating parameters of the driving apparatus based on the deviation may be implemented based on the prior art. The embodiments of the present disclosure impose no limitation on the specific implementation of adjusting the operating parameters of the driving apparatus based on the deviation.

When the currently determined deviation decreases compared to the deviation determined in the previous execution of step S32, it indicates that the deviation can be reduced by continuing to use the preset operating parameters to move the ultrasound probe. When the currently determined deviation is still greater than the first preset value, the driving apparatus is continuously controlled to operate according to the preset operating parameters to drive the ultrasound probe to move.

After each deviation determination, the currently determined deviation is compared with the previously determined deviation, and the operating parameters of the driving apparatus are adjusted based on a comparison result. In this case, as the number of movements of the ultrasound probe increases, the deviation gradually decreases until the deviation falls below or equals the first preset value, at which point control of the driving apparatus is terminated.

In this way, the manner in which the processor controls the driving apparatus becomes more flexible.

The embodiments of the present disclosure further provide an ultrasound probe assembly including an ultrasound probe, a driving apparatus, a grid plate, and at least one puncture needle, the at least one puncture needle being inserted into at least one reference point on the grid plate.

The ultrasound probe is configured to acquire an ultrasound image including an image of the at least one puncture needle and a two-dimensional coordinate system, the two-dimensional coordinate system including at least one coordinate point corresponding to at least one reference point.

The driving apparatus is configured to drive the ultrasound probe to move so as to cause a deviation between the image of the at least one puncture needle and the at least one coordinate point to be less than or equal to a first preset value.

For connection relationships among components in the ultrasound probe assembly, refer to FIG. 1. The functions of the components have been described above, and details are not described again here.

In one possible implementation, the driving apparatus includes at least one of a translation motor and a rotation motor, the translation motor being configured to drive the ultrasound probe to translate, and the rotation motor being configured to drive the ultrasound probe to rotate.

For example, the driving apparatus may include at least one of a translation motor and a rotation motor. The translation motor is configured to drive the ultrasound probe to translate, and the rotation motor is configured to drive the ultrasound probe to rotate. Further, if a single translation motor can drive the ultrasound probe to translate along one line, and the application scenario requires driving the ultrasound probe to translate along a plurality of lines, a plurality of translation motors may be provided. If a single rotation motor can drive the ultrasound probe to rotate about one axis, and the application scenario requires driving the ultrasound probe to rotate about a plurality of axes, a plurality of rotation motors may be provided. The embodiments of the present disclosure impose no limitation on how many translation motors and rotation motors are specifically included in the driving apparatus.

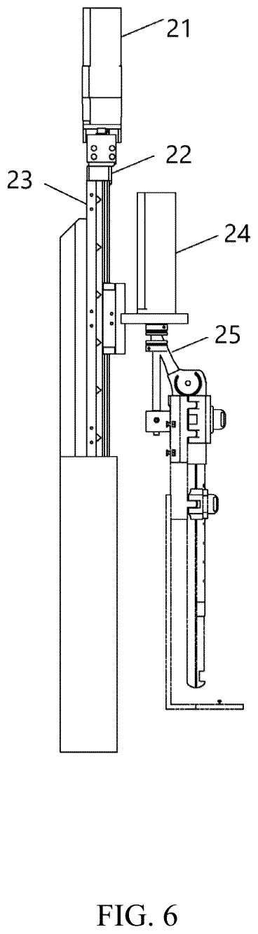

FIG. 6 shows a schematic diagram of the structure of a driving apparatus according to an embodiment of the present disclosure.

In one possible implementation, the driving apparatus further includes a guide rail, a first connecting member, and a second connecting member, the translation motor being connected to the guide rail by means of the first connecting member, the rotation motor being fixed to a sliding block on the guide rail, and the rotation motor being connected to the ultrasound probe by means of the second connecting member.

The translation motor causes the rotation motor, the second connecting member, and the ultrasound probe to translate by means of translating the sliding block.

The rotation motor causes the ultrasound probe to rotate by means of rotating the second connecting member.

For example, as shown in FIG. 6, the driving apparatus may include a translation motor 21, a first connecting member 22, a guide rail 23, a rotation motor 24, and a second connecting member 25. The first connecting member may be a screw nut, and the second connecting member may be a bearing. The embodiments of the present disclosure impose no limitation on the specific structures of the first connecting member and the second connecting member. The translation motor may be connected to the guide rail by means of the first connecting member and the sliding block provided on the guide rail. During operation, the translation motor can drive the sliding block to move on the guide rail. The rotation motor is fixed to the sliding block, and when the sliding block moves, the rotation motor moves together with the sliding block. The rotation motor is further connected to the ultrasound probe by means of the second connecting member. Therefore, when the sliding block moves, the rotation motor, the second connecting member, and the ultrasound probe can move together with the sliding block. During operation, the rotation motor may rotate the ultrasound probe by means of the second connecting member. In this way, the driving apparatus can support the translation and rotation of the ultrasound probe.

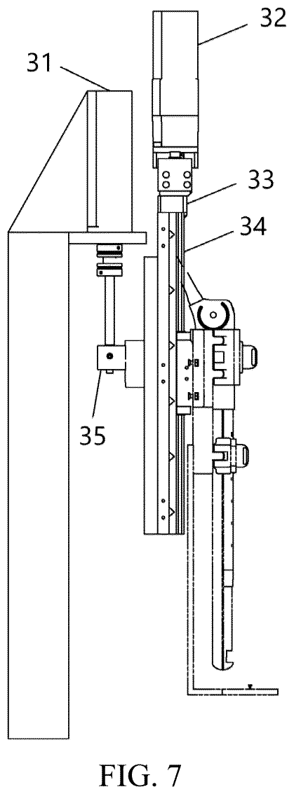

FIG. 7 shows a schematic diagram of the structure of a driving apparatus according to an embodiment of the present disclosure.

In one possible implementation, the driving apparatus further includes a guide rail, a third connecting member, and a fourth connecting member, the rotation motor being connected to the guide rail by means of the third connecting member, the translation motor being connected to the guide rail by means of the fourth connecting member, and the ultrasound probe being fixed to a sliding block on the guide rail.

The rotation motor causes the guide rail, the fourth connecting member, the sliding block, and the ultrasound probe to rotate by means of rotating the third connecting member.

The translation motor causes the ultrasound probe to translate by means of translating the sliding block.

For example, as shown in FIG. 7, the driving apparatus may include a rotation motor 31, a translation motor 32, a fourth connecting member 33, a guide rail 34, and a third connecting member 35. The fourth connecting member may be a screw nut, and the third connecting member may be a bearing. The embodiments of the present disclosure impose no limitation on the specific structures of the fourth connecting member and the third connecting member. The rotation motor may be connected to the guide rail by means of the third connecting member and the sliding block provided on the guide rail. The guide rail is further connected to the translation motor by means of the fourth connecting member, and the sliding block is fixed to the rotation motor. During operation, the rotation motor may cause the guide rail to rotate by means of the third connecting member. As a result, the sliding block, the fourth connecting member, the translation motor, and the ultrasound probe also rotate together. The translation motor may drive the ultrasound probe to move by means of causing the sliding block to move on the guide rail. In this way, the driving apparatus can support the translation and rotation of the ultrasound probe.

The above takes the driving apparatus including a single translation motor and a single rotation motor as an example. As described above, the driving apparatus may also be provided with more translation motors and rotation motors, to achieve translation and rotation in more directions. The embodiments of the present disclosure impose no limitation on the specific structure of the driving apparatus.

In an actual application scenario, the structure of the driving apparatus may be set according to the requirements of the application scenario, thereby increasing the flexibility of the structure of the driving apparatus.

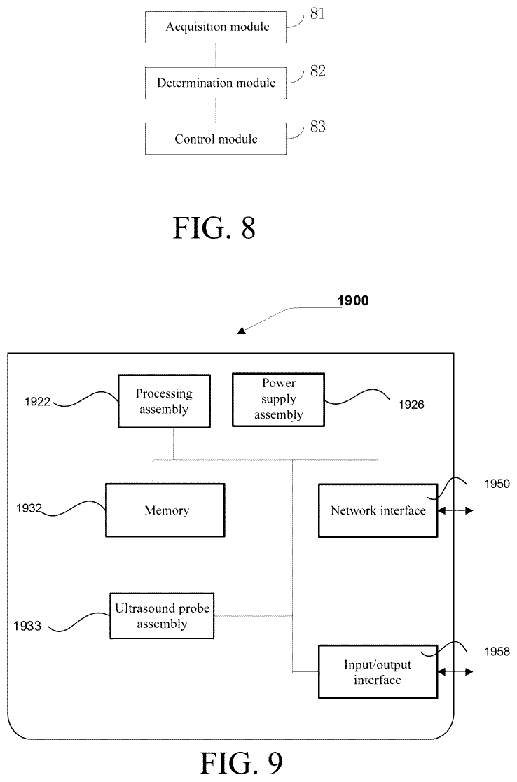

The embodiments of the present disclosure further provide a calibration apparatus for an ultrasound probe assembly. FIG. 8 shows a schematic diagram of the structure of a calibration apparatus for an ultrasound probe assembly according to an embodiment of the present disclosure.

As shown in FIG. 8, in one possible implementation, the ultrasound probe assembly includes an ultrasound probe, a driving apparatus, a grid plate, and at least one puncture needle. The at least one puncture needle is inserted into at least one reference point on the grid plate. The apparatus includes:

An acquisition module 81, configured to acquire, using the ultrasound probe, an ultrasound image including an image of the at least one puncture needle and a two-dimensional coordinate system, the two-dimensional coordinate system including at least one coordinate point corresponding to the at least one reference point;

A determination module 82, configured to determine a deviation between the image of the at least one puncture needle and the at least one coordinate point; and

A control module 83, configured to control, in response to the deviation being greater than a first preset value, the driving apparatus to drive the ultrasound probe to move the deviation to be less than or equal to the first preset value.

In one possible implementation, the coordinates of the coordinate point corresponding to each reference point in the two-dimensional coordinate system are associated with the position of the reference point on the grid plate.

In one possible implementation, the coordinates of the coordinate point corresponding to each reference point in the two-dimensional coordinate system are inputted by a user.

In one possible implementation, determining a deviation between the image of the at least one puncture needle and the at least one coordinate point includes: identifying the image of at least one puncture needle from the ultrasound image; and determining the deviation between the image of the at least one puncture needle and the at least one coordinate point according to a linear distance between the image of the at least one puncture needle and the coordinate point corresponding thereto in the two-dimensional coordinate system.

In one possible implementation, a manner in which the driving apparatus drives the ultrasound probe to move includes at least one of translation and rotation.

In one possible implementation, controlling, in response to the deviation being greater than a first preset value, the driving apparatus to drive the ultrasound probe to move includes: determining operating parameters of the driving apparatus according to the coordinates of the image of each puncture needle and the coordinate point corresponding thereto in the two-dimensional coordinate system, wherein the operating parameters include at least one of a set of translation parameters and a set of rotation parameters, the translation parameters including a translation direction and a translation distance, and the rotation parameters including a rotation direction and a rotation angle; controlling the driving apparatus to drive, according to the operating parameters, the ultrasound probe to move; and determining again the deviation between the image of the at least one puncture needle and the at least one coordinate point.

In one possible implementation, controlling, in response to the deviation being greater than a first preset value, the driving apparatus to drive the ultrasound probe to move includes: controlling the driving apparatus to drive, according to preset operating parameters, the ultrasound probe to move, wherein the operating parameters include at least one of a set of translation parameters and a set of rotation parameters, the translation parameters including a translation direction and a translation distance, and the rotation parameters including a rotation direction and a rotation angle; determining the deviation again; adjusting, when the deviation has increased, at least one of the preset operating parameters, and controlling the driving apparatus to drive, according to the adjusted operating parameter, the ultrasound probe to move; and maintaining, when the deviation has decreased but remains greater than the first preset value, the preset operating parameters unchanged, and continuing to control the driving apparatus to drive, according to the preset operating parameters, the ultrasound probe to move.

In one possible implementation, the at least one puncture needle includes a first puncture needle and a second puncture needle, the at least one reference point includes a first reference point and a second reference point, and the at least one coordinate point includes a first coordinate point and a second coordinate point.

In one possible implementation, the at least one puncture needle includes a first puncture needle and a second puncture needle, and the at least one coordinate point includes a first coordinate point and a second coordinate point; and determining operating parameters of the driving apparatus according to the coordinates of the image of each puncture needle and the coordinate point corresponding thereto in the two-dimensional coordinate system includes: calculating a linear distance between the image of the first puncture needle and the image of the second puncture needle in the two-dimensional coordinate system according to the coordinates of the image of the first puncture needle and the image of the second puncture needle in the two-dimensional coordinate system, to obtain a first value; calculating a linear distance between the first coordinate point and the second coordinate point in the two-dimensional coordinate system according to the coordinates of the first coordinate point and the second coordinate point in the two-dimensional coordinate system, to obtain a second value; determining a translation distance according to a difference between the second value and the first value; and determining a translation direction according to a magnitude relationship between the second value and the first value.

In one possible implementation, the at least one puncture needle includes a first puncture needle and a second puncture needle, and the at least one coordinate point includes a first coordinate point and a second coordinate point; and determining operating parameters of the driving apparatus according to the coordinates of the image of each puncture needle and the coordinate point corresponding thereto in the two-dimensional coordinate system includes: determining a first connecting line between the image of the first puncture needle and the image of the second puncture needle in the two-dimensional coordinate system according to the coordinates of the image of the first puncture needle and the image of the second puncture needle in the two-dimensional coordinate system; determining a second connecting line between the first coordinate point and the second coordinate point in the two-dimensional coordinate system according to the coordinates of the first coordinate point and the second coordinate point in the two-dimensional coordinate system; determining a rotation angle according to an included angle between the first connecting line and the second connecting line; and determining a rotation direction according to a magnitude relationship between slopes of the first connecting line and the second connecting line.

In some embodiments, a function of the apparatus provided in the embodiments of the present disclosure or the modules included in the apparatus may be configured to perform the method described in the foregoing method embodiments. For a specific implementation thereof, reference may be made to descriptions of the foregoing method embodiments. For brevity, details are not described herein again.

The embodiments of the present disclosure further provide an ultrasound imaging system. In one possible implementation, the system includes:

A processor;

A memory, configured to store instructions executable by the processor; and

An ultrasound probe assembly, including an ultrasound probe, a driving apparatus, a grid plate, and at least one puncture needle, the at least one puncture needle being inserted into at least one reference point on the grid plate.

The processor is configured to implement the described method when executing the instructions stored in the memory.

FIG. 9 shows a schematic diagram of the structure of an ultrasound imaging system according to an embodiment of the present disclosure. As shown in FIG. 9, the ultrasound imaging system may be provided as a server or a terminal device. Referring to FIG. 9, the ultrasound imaging system 1900 includes a processing assembly 1922 that further includes one or more processors, and a memory resource represented by a memory 1932, used for storing instructions executable by the processing assembly 1922, such as an application. The application stored in the memory 1932 can include one or more modules each corresponding to a set of instructions. In addition, the processing assembly 1922 is configured to execute instructions to execute the above method.

The ultrasound imaging system 1900 may further include a power supply assembly 1926, configured to execute power management for the ultrasound imaging system 1900, a wired or wireless network interface 1950, configured to connect the ultrasound imaging system 1900 to a network, and an input/output interface (I/O interface) 1958. The ultrasound imaging system 1900 may operate based on an operating system stored in the memory 1932, for example, Windows Server™, Mac OS X™, Unix™, Linux™, or FreeBSD™.

In an exemplary embodiment, a non-volatile computer-readable storage medium is also provided, such as the memory 1932 including computer program instructions that are executable by the processing assembly 1922 of the ultrasonic imaging system 1900 to complete the above method.

For the structure and function of the ultrasound probe assembly 1933, refer to FIG. 1 and related descriptions.

In one possible implementation, the ultrasound probe assembly is further connected to a robotic arm, and the processor is further configured to execute the instructions stored in the memory to implement:

Maintaining, after a deviation is less than or equal to a first preset value, a relative position between the ultrasound probe and the grid plate unchanged, and controlling the robotic arm to move the ultrasound probe assembly to an operating table.

For example, as described in relation to FIG. 2, the ultrasound probe assembly may be further connected to a robotic arm, and the robotic arm configured to move the ultrasound probe assembly. By way of example, after the deviation is less than or equal to the first preset value, that is, after the calibration of the ultrasound probe is completed, the ultrasound probe assembly may be moved to the operating table to prepare for an interventional procedure. It should be noted that, during the movement of the ultrasound probe assembly, the relative position between the ultrasound probe and the grid plate should be maintained unchanged, such that the ultrasound probe assembly is still maintained in a state of completed calibration after being moved to the operating table.

In this way, the ultrasound probe assembly can be put into use more quickly after calibration.

In one possible implementation, the processor is further configured to execute instructions stored in the memory to implement:

Identifying, during an interventional procedure, a region of interest in an intraoperative ultrasound image acquired by the ultrasound probe, the intraoperative ultrasound image including an image of a tissue to be imaged and a two-dimensional coordinate system, the two-dimensional coordinate system including a target coordinate point; and

Controlling the driving apparatus to drive, according to the coordinates of the region of interest and the target coordinate point in the two-dimensional coordinate system included in the intraoperative ultrasound image, the ultrasound probe to move.

For example, in an interventional procedure, it is necessary to first perform ultrasound imaging on a tissue to be imaged, and then determine a lesion location. Therefore, an intraoperative ultrasound image may include an image of the tissue to be imaged.

To facilitate observation, it is necessary to make the lesion location as central as possible within the intraoperative ultrasound image. In this regard, the intraoperative ultrasound image may further include a two-dimensional coordinate system, the two-dimensional coordinate system including a target coordinate point, e.g., a center point of the intraoperative ultrasound image.

Since the processor has the capability to control the driving apparatus to drive the ultrasound probe to move, the processor may identify a region of interest in the intraoperative ultrasound image acquired by the ultrasound probe, e.g., a region in which a lesion is located or an important anatomical structure such as a prostate, and control the driving apparatus to drive the ultrasound probe to move so as to cause a deviation between the region of interest and the target coordinate point to be less than or equal to a second preset value. The second preset value may be set according to the requirements of application scenarios, and may be the same as or different from the first preset value. The embodiments of the present disclosure impose no limitation on the specific value of the second preset value.

In this way, the driving apparatus can be used not only during calibration but also during a procedure, thereby improving the utilization rate of the driving apparatus.

In one possible implementation, controlling the driving apparatus to drive, according to the coordinates of the region of interest and the target coordinate point in the two-dimensional coordinate system included in the intraoperative ultrasound image, the ultrasound probe to move includes:

Determining operating parameters of the driving apparatus according to the coordinates of the region of interest and the target coordinate point in the two-dimensional coordinate system included in the intraoperative ultrasound image, the operating parameters including at least one of a set of translation parameters and a set of rotation parameters, the translation parameters including a translation direction and a translation distance, and the rotation parameters including a rotation direction and a rotation angle; and

Causing the driving apparatus to operate according to the operating parameters.

For example, according to the coordinates of the region of interest and the target coordinate point in the two-dimensional coordinate system included in the intraoperative ultrasound image, the specific manner of controlling the driving apparatus to drive the ultrasound probe to move may be the same as the manner of controlling the driving apparatus to drive the ultrasound probe to move when the ultrasound probe is calibrated. A center point of the region of interest is regarded as the image of the puncture needle, and the target coordinate point is regarded as the coordinate point corresponding to the puncture needle. That is, the operating parameters of the driving apparatus may be determined according to the coordinates of the region of interest and the target coordinate point in the two-dimensional coordinate system included in the intraoperative ultrasound image, the operating parameters including at least one of a set of translation parameters and a set of rotation parameters, the translation parameters including a translation direction and a translation distance, and the rotation parameters including a rotation direction and a rotation angle; and the driving apparatus is caused to operate according to the operating parameters. Exemplary manners of determining the driving parameters have been described above, and details will not be described again here.

In this way, the accuracy of intraoperative control of the movement of the ultrasound probe can be ensured.

In one possible implementation, the processor is further configured to execute instructions stored in the memory to implement:

Causing, after controlling the driving apparatus to drive the ultrasound probe to move, the ultrasound probe to reacquire an intraoperative ultrasound image, and identifying a region of interest in the reacquired intraoperative ultrasound image;

Determining a distance between a center point of the region of interest and the target coordinate point in a two-dimensional coordinate system included in the reacquired intraoperative ultrasound image; and

Continuing to control, when the distance is greater than a second preset value, the driving apparatus to drive the ultrasound probe to move.

For example, similar to the manner of controlling the driving apparatus during calibration of the ultrasound probe, there may be errors in the driving of the ultrasound probe by the driving apparatus. Therefore, in an interventional procedure, after the driving apparatus is controlled to drive the ultrasound probe to move, the ultrasound probe may be caused to reacquire an intraoperative ultrasound image, the region of interest in the reacquired intraoperative ultrasound image may be identified, the distance between the center point of the region of interest and the target coordinate point in the two-dimensional coordinate system included in the reacquired intraoperative ultrasound image may be determined, and, when the distance is greater than the second preset value, the driving apparatus may be controlled to continue driving the ultrasound probe to move.

In this way, the accuracy of the driving apparatus intraoperatively driving the ultrasound probe can be improved.

The embodiments of the present disclosure further provide a computer-readable storage medium, having computer program instructions stored thereon, wherein the computer program instructions, when executed by a processor, implement the above method. The computer-readable storage medium may be a volatile or non-volatile computer-readable storage medium.

The embodiments of the present disclosure further provide a computer program product, including computer-readable code or a non-volatile computer-readable storage medium bearing computer-readable code, wherein when the computer-readable code is run in a processor in an electronic device, the processor in the electronic device performs the above method.

The present disclosure may be a system, a method and/or a computer program product. The computer program product may include a computer-readable storage medium, having computer-readable program instructions thereon for causing a processor to implement various aspects of the present disclosure.

The computer-readable storage medium can be a tangible device that can hold and store instructions used by an instruction execution device. The computer-readable storage medium may be, for example, but is not limited to, an electrical storage device, a magnetic storage device, an optical storage device, an electromagnetic storage device, a semiconductor memory device, or any suitable combination thereof. More specific examples (a non-exhaustive list) of the computer-readable storage medium include: a portable computer disk, a hard disk, a random access memory (RAM), a read-only memory (ROM), a erasable programmable read-only memory (EPROM or flash memory), a static random access memory (SRAM), a portable compact disc read-only memory (CD ROM), a digital versatile disk (DVD), a memory stick, a floppy disk, mechanical coding equipment, such as a punch card having instructions stored thereon or a structure of bumps within recessions, and any suitable combination thereof. The computer-readable storage medium used herein is not interpreted as transient signals themselves, such as radio waves or other freely propagated electromagnetic waves, electromagnetic waves propagated through a waveguide or other transmission media (e.g., light pulses passing through a fiber optic cable), or electrical signals transmitted through electric wires.

The computer-readable program instructions described herein may be downloaded from a computer-readable storage medium to various computing/processing devices or downloaded to an external computer or external storage device via a network such as the Internet, a local area network, a wide area network and/or a wireless network. The network may include copper transmission cables, fiber transmission, wireless transmission, routers, firewalls, switches, gateway computers and/or edge servers. A network adapter card or a network interface in each computing/processing device receives computer-readable program instructions from the network and forwards the computer-readable program instructions, for storing the same in a computer-readable storage medium in each computing/processing device.

Computer program instructions for executing the operations of the present disclosure can be assembly instructions, instruction set architecture (ISA) instructions, machine instructions, machine related instructions, microcode, firmware instructions, state setting data, or source code or object code written in any combination of one or more programming languages, the programming language including object oriented programming languages such as Smalltalk, C++, and the like, and conventional procedural programming languages such as the “C” language or similar programming languages. The computer-readable program instructions can be executed entirely or partly on a user computer, executed as a stand-alone software package, executed partly on a user computer and partly on a remote computer, or executed entirely on a remote computer or server. In the case of a remote computer, the remote computer may be connected to a user computer through any kind of network, including a local area network (LAN) or a wide area network (WAN), or connected to an external computer (for example, through the Internet, using an Internet service provider). In some embodiments, an electronic circuit, for example, a programmable logic circuit, a field-programmable gate array (FPGA), or a programmable logic array (PLA), may execute the computer-readable program instructions by utilizing state information of the computer-readable program instructions to personalize the electronic circuit, in order to implement various aspects of the present disclosure.

The aspects of the present disclosure are described herein with reference to the flowcharts and/or block diagrams of the methods, apparatuses (systems), and computer program products according to the embodiments of the present disclosure. It should be understood that each block of the flowcharts and/or block diagrams and combinations of various blocks in the flowcharts and/or block diagrams can be implemented by computer-readable program instructions.

These computer-readable program instructions may be provided to a processor of a general-purpose computer, a special-purpose computer, or other programmable data processing apparatuses, to produce a machine, so that these instructions, when executed by the processor of the computer or other programmable data processing apparatuses, produce an apparatus for implementing the functions/actions specified in one or more blocks of the flowcharts and/or block diagrams. Also, these computer-readable program instructions may be stored in a computer-readable storage medium. These instructions cause a computer, a programmable data processing apparatus, and/or other devices to work in a specific manner. Thus, the computer-readable medium storing the instructions includes an artifact, including instructions that implement various aspects of the functions/actions specified in one or more the flowcharts and/or block diagrams.

The computer-readable program instructions may also be loaded onto a computer, other programmable data processing apparatuses, or other devices, such that the computer, other programmable data processing apparatuses or other devices execute a series of operational steps, to generate a computer-implemented process, such that the functions/actions specified in one or more of the flowcharts and/or block diagrams are implemented by the instructions executed on the computer, other programmable data processing apparatuses, or other devices.

The flowcharts and block diagrams in the accompanying drawings illustrate system architectures, functions, and operations of possible implementations of the system, method, and computer program product according to a plurality of embodiments of the present disclosure. In this regard, each block in the flowcharts or block diagrams may represent a portion of a module, program segment, or instruction that contains one or more executable instructions for implementing the specified logical functions. In some alternative implementations, the functions denoted in the blocks can also occur in a different order than that illustrated in the drawings. For example, two consecutive blocks can actually be executed substantially in parallel, and sometimes can also be executed in a reverse order, depending upon the functions involved. It should also be noted that each block of the block diagrams and/or flowcharts, and combinations of blocks in the block diagrams and/or flowcharts can be implemented in a dedicated hardware-based system that executes the specified function or action, or can be implemented by a combination of dedicated hardware and computer instructions.

The embodiments of the present disclosure have been described above. The foregoing description is illustrative rather than limiting, and is not limited to the disclosed embodiments. Many modifications and variations are apparent to those of ordinary skill in the art without departing from the scope and spirit of the embodiments illustrated. The selection of terms used herein is intended to best explain the principles, practical applications, or improvements to the technologies in the market of the embodiments, or to enable others of ordinary skill in the art to understand the embodiments disclosed herein.

What is claimed is:

1. A method for calibrating an ultrasound probe assembly, the ultrasound probe assembly comprising an ultrasound probe, a driving apparatus, a grid plate, and at least one puncture needle, and the at least one puncture needle inserted into at least one reference point on the grid plate, the method comprising:
obtaining, using the ultrasound probe, an ultrasound image comprising an image of the at least one puncture needle and a two-dimensional coordinate system, the two-dimensional coordinate system comprising at least one coordinate point corresponding to the at least one reference point;
determining a deviation between the image of the at least one puncture needle and the at least one coordinate point; and
controlling, based on the deviation being greater than a first preset value, the driving apparatus to drive the ultrasound probe to move so as to cause the deviation to be less than or equal to the first preset value.

2. The method according to claim 1, wherein the coordinates of the coordinate point corresponding to each reference point in the two-dimensional coordinate system are associated with the position of the reference point on the grid plate.

3. The method according to claim 1, wherein the coordinates of the coordinate point corresponding to each reference point in the two-dimensional coordinate system are inputted by a user.

4. The method according to claim 1, wherein
the determining a deviation between the image of the at least one puncture needle and the at least one coordinate point comprises:
identifying the image of the at least one puncture needle from the ultrasound image; and
determining the deviation between the image of the at least one puncture needle and the at least one coordinate point according to a linear distance between the image of the at least one puncture needle and the coordinate point corresponding thereto in the two-dimensional coordinate system.

5. The method according to claim 1, wherein a manner in which the driving apparatus drives the ultrasound probe to move comprises at least one of translation and rotation.

6. The method according to claim 1, wherein
the controlling, in response to the deviation being greater than a first preset value, the driving apparatus to drive the ultrasound probe to move comprises:
determining operating parameters of the driving apparatus according to the coordinates of the image of each puncture needle and the coordinate point corresponding thereto in the two-dimensional coordinate system, wherein the operating parameters comprise at least one of a set of translation parameters and a set of rotation parameters, the translation parameters comprising a translation direction and a translation distance, and the rotation parameters comprising a rotation direction and a rotation angle;
controlling the driving apparatus to drive, according to the operating parameters, the ultrasound probe to move; and
determining again the deviation between the image of the at least one puncture needle and the at least one coordinate point.

7. The method according to claim 1, wherein the controlling, in response to the deviation being greater than a first preset value, the driving apparatus to drive the ultrasound probe to move comprises:
controlling the driving apparatus to drive, according to preset operating parameters, the ultrasound probe to move, the operating parameters comprising at least one of a set of translation parameters and a set of rotation parameters, the translation parameters comprising a translation direction and a translation distance, and the rotation parameters comprising a rotation direction and a rotation angle;
determining the deviation again;
adjusting, when the deviation has increased, at least one of the preset operating parameters, and controlling the driving apparatus to drive, according to the adjusted operating parameter, the ultrasound probe to move; and
maintaining, when the deviation has decreased but remains greater than the first preset value, the preset operating parameters unchanged, and continuing to control the driving apparatus to drive, according to the preset operating parameters, the ultrasound probe to move.

8. The method according to claim 1, wherein the at least one puncture needle comprises a first puncture needle and a second puncture needle, the at least one reference point comprises a first reference point and a second reference point, and the at least one coordinate point comprises a first coordinate point and a second coordinate point.

9. The method according to claim 6, wherein the at least one puncture needle comprises a first puncture needle and a second puncture needle, and the at least one coordinate point comprises a first coordinate point and a second coordinate point;
and the determining operating parameters of the driving apparatus according to the coordinates of the image of each puncture needle and the coordinate point corresponding thereto in the two-dimensional coordinate system comprises:
calculating a linear distance between the image of the first puncture needle and the image of the second puncture needle in the two-dimensional coordinate system according to the coordinates of the image of the first puncture needle and the image of the second puncture needle in the two-dimensional coordinate system, to obtain a first value;
calculating a linear distance between the first coordinate point and the second coordinate point in the two-dimensional coordinate system according to the coordinates of the first coordinate point and the second coordinate point in the two-dimensional coordinate system, to obtain a second value;
determining the translation distance according to a difference between the second value and the first value; and
determining the translation direction according to a magnitude relationship between the second value and the first value.

10. The method according to claim 6, wherein the at least one puncture needle comprises a first puncture needle and a second puncture needle, and the at least one coordinate point comprises a first coordinate point and a second coordinate point;
and the determining operating parameters of the driving apparatus according to the coordinates of the image of each puncture needle and the coordinate point corresponding thereto in the two-dimensional coordinate system comprises:
determining a first connecting line between the image of the first puncture needle and the image of the second puncture needle in the two-dimensional coordinate system according to the coordinates of the image of the first puncture needle and the image of the second puncture needle in the two-dimensional coordinate system;
determining a second connecting line between the first coordinate point and the second coordinate point in the two-dimensional coordinate system according to the coordinates of the first coordinate point and the second coordinate point in the two-dimensional coordinate system;
determining the rotation angle according to an included angle between the first connecting line and the second connecting line; and
determining the rotation direction according to a magnitude relationship between slopes of the first connecting line and the second connecting line.

11. An ultrasound probe assembly, comprising:
an ultrasound probe;
a driving apparatus;
a grid plate; and
at least one puncture needle;
wherein the at least one puncture needle is configured to be inserted into at least one reference point on the grid plate,
wherein the ultrasound probe is configured to acquire an ultrasound image comprising an image of the at least one puncture needle and a two-dimensional coordinate system, the two-dimensional coordinate system comprising at least one coordinate point corresponding to the at least one reference point, and
wherein the driving apparatus is configured to drive the ultrasound probe to move so as to cause a deviation between the image of the at least one puncture needle and the at least one coordinate point to be less than or equal to a first preset value.

12. The ultrasound probe assembly according to claim 11, wherein the driving apparatus comprises at least one of a translation motor and a rotation motor, the translation motor being configured to drive the ultrasound probe to translate, and the rotation motor being configured to drive the ultrasound probe to rotate.

13. The ultrasound probe assembly according to claim 12, wherein the driving apparatus further comprises a guide rail, a first connecting member, and a second connecting member, the translation motor being connected to the guide rail by means of the first connecting member, the rotation motor being fixed to a sliding block on the guide rail, and the rotation motor being connected to the ultrasound probe by means of the second connecting member;
the translation motor causes the rotation motor, the second connecting member, and the ultrasound probe to translate by means of translating the sliding block; and
the rotation motor causes the ultrasound probe to rotate by means of rotating the second connecting member.

14. The ultrasound probe assembly according to claim 12, wherein the driving apparatus further comprises a guide rail, a third connecting member, and a fourth connecting member, the rotation motor being connected to the guide rail by means of the third connecting member, the translation motor being connected to the guide rail by means of the fourth connecting member, and the ultrasound probe being fixed to a sliding block on the guide rail;
the rotation motor causes the guide rail, the fourth connecting member, the sliding block, and the ultrasound probe to rotate by means of rotating the third connecting member; and
the translation motor causes the ultrasound probe to translate by means of translating the sliding block.

15. An ultrasound imaging system, comprising:
a processor;
a memory, configured to store instructions executable by the processor; and
an ultrasound probe assembly, comprising an ultrasound probe, a driving apparatus, a grid plate, and at least one puncture needle, the at least one puncture needle being inserted into at least one reference point on the grid plate;
wherein the processor is configured to execute the instructions to:
obtain an ultrasound image comprising an image of the at least one puncture needle and a two-dimensional coordinate system, the two-dimensional coordinate system comprising at least one coordinate point corresponding to the at least one reference point;
determine a deviation between the image of the at least one puncture needle and the at least one coordinate point; and
control, based on the deviation being greater than a first preset value, the driving apparatus to drive the ultrasound probe to move so as to cause the deviation to be less than or equal to the first preset value.

16. The system according to claim 15, wherein the processor is further configured to execute the instructions stored in the memory to implement:
identifying, during an interventional procedure, a region of interest in an intraoperative ultrasound image acquired by the ultrasound probe, the intraoperative ultrasound image comprising an image of a tissue to be imaged and a two-dimensional coordinate system, and the two-dimensional coordinate system comprising a target coordinate point; and
controlling, according to the coordinates of the region of interest and the target coordinate point in the two-dimensional coordinate system comprised by the intraoperative ultrasound image, the driving apparatus to drive the ultrasound probe to move.

17. The system according to claim 16, wherein the controlling, according to the coordinates of the region of interest and the target coordinate point in the two-dimensional coordinate system comprised by the intraoperative ultrasound image, the driving apparatus to drive the ultrasound probe to move comprises:
determining operating parameters of the driving apparatus according to the coordinates of the region of interest and the target coordinate point in the two-dimensional coordinate system comprised by the intraoperative ultrasound image, wherein the operating parameters comprise at least one of a set of translation parameters and a set of rotation parameters, the translation parameters comprising a translation direction and a translation distance, and the rotation parameters comprising a rotation direction and a rotation angle; and
causing the driving apparatus to operate according to the operating parameters.

18. The system according to claim 16, wherein the processor is further configured to execute the instructions stored in the memory to implement:
causing, after controlling the driving apparatus to drive the ultrasound probe to move, the ultrasound probe to reacquire an intraoperative ultrasound image, and identifying a region of interest in the reacquired intraoperative ultrasound image;
determining a distance between a center point of the region of interest and the target coordinate point in a two-dimensional coordinate system comprised by the reacquired intraoperative ultrasound image; and
continuing to control, when the distance is greater than a second preset value, the driving apparatus to drive the ultrasound probe to move.
